(19) United States
(12) Patent Application Publication
(10) Pub. No.: US 2017/0072106 A1
(43) Pub. Date: Mar. 16, 2017

(54) COMPOUND FOR STIMULATING BONE FORMATION

(71) Applicant: Elos Medtech Timmersdala AB, Timmersdala (SE)

(72) Inventor: Hakan Nygren, Billdal (SE)

(21) Appl. No.: 15/123,308

(22) PCT Filed: Mar. 2, 2015
(86) PCT No.: PCT/SE2015/050235

§ 371 Date: Sep. 2, 2016

(30) Foreign Application Priority Data
Mar. 3, 2014 (SE) 1450235-5

(51) Int. Cl. A61L 27/54; A61L 27/32; A61L 27/12; A61J 1/06; A61L 27/20; C04B 28/34; C04B 28/30; A61L 27/30; A61L 27/04

(52) U.S. Cl. CPC A61L 27/54; A61L 27/306; A61L 27/32; A61L 27/12; A61L 27/047; A61L 27/20; C04B 28/344; C04B 28/30; A61J 1/067; A61L 2430/02; A61L 2300/102; A61L 2420/06; C04B 2111/00836

(57) ABSTRACT

Compounds for stimulating bone formation are provided. The compound consists of a paste or similar material with viscous properties that can be coated on and spread over bone tissue and other surfaces of interest, wherein the compound contains Mg and at least one of the elements Ca, Mn and Zn, and wherein Mg constitutes at least 75 percent by weight of the total quantity of Mg, Ca, Mn and Zn in the compound. Methods for stimulating bone formation, medical kits, and methods for manufacturing the compound are also provided.

### TECHNICAL AREA

Compound for the specific local stimulation of bone formation into compact bone and trabecular bone.

### BACKGROUND

Weak bone, brittle bone or osteoporosis, is a common condition in an aging population with a prevalence of approx. 30% in women aged 70-80. Bone defects, for example as a consequence of radiotherapy, are also a clinical condition where there is a need for an increase in the formation of new differentiated bone tissue.

There are several types of pharmaceutical that can affect bone tissue and mineralisation when generally administered. Biphosphonates act via two different mechanisms, both by binding to hydroxyapatite, which reduces the turnover of the mineral and via uptake in the osteoclast's mitochondria with subsequent cell death. Strontium ranelate acts via its effect on bone-forming cells and results in an increased bone density in trabecular bone, but does not affect compact cortical bone. Pharmaceuticals that are generally administered have adverse effects, which can be very troublesome at times. For this reason, there is a wish for locally acting agents to influence bone formation and bone turnover.

Among the agents with local effect, are bone morphogenetic proteins that act by stimulating the differentiation of bone-forming cells from mesenchymal stem cells. The clinical effect of the therapy is moderate (4% improvement of failed fracture healing in the tibia). Fluoride ions act by binding to hydroxyapatite and reducing the turnover of the mineral.

At the present, it seems that no method/compound is known that stimulates bone formation, which results in the specific simulation of the formation of compact, cortical bone.

### INVENTION

One aspect of the invention is to provide a compound/preparation that provides improved bone formation, in particular, of compact cortical bone.

The invention concerns a compound for stimulating bone formation, wherein the compound consists of a paste or similar substance with viscous properties that can be coated on and spread over bone tissue and other surfaces of interest, wherein the compound contains Mg and at least one of the elements Ca, Mn and Zn, and wherein Mg constitutes at least 75 percent by weight of the total quantity of Mg, Ca, Mn and Zn in the compound.

The invention is based on the knowledge that magnesium (Mg) has a specially beneficial effect regarding the stimulation of bone formation. The compound according to the invention has a high proportion of Mg for this reason.

The invention is also based on the knowledge that a paste or similar substance with viscous properties is very suitable for local application in order to attain thereby a good local effect without the effects that pharmaceuticals that are generally administered can give rise to.

In order to study the effect of Mg ions on the bone tissue, implants of a compound were made in the bone marrow. A sterile preparation/compound of MgO, which was made into a paste using saline solution, was implanted in marrow cavities in the bone and left to act. Thereby, a specific effect on the thickness of the compact, cortical bone wall was discovered. The effect was observed 3 weeks after the insertion of the agent. After 2 weeks, there is no measurable effect. On analysis using a histological method, a statistically significant (p<0.001) stimulation of the thickness of the cortical compact bone wall was found. This is an important discovery, since it is this bone wall that takes up the forces when the bone is loaded. A strong bone wall implies an increased protection against bone fracture.

Furthermore, the invention is based on the knowledge that the presence of calcium (Ca), manganese (Mn) and/or zinc (Zn) in the compound increases the beneficial effect of the magnesium regarding stimulation of bone formation. For this reason, the compound according to the invention contains a controlled quantity of at least one of these elements, preferably of several.

The mechanism for how Ca, Mn and Zn increase magnesium's beneficial effect has not been fully studied. Nevertheless, there are indications that the mechanism underlying this effect is the activation of growth factors, primarily VEGF, which promote the growth of new blood vessels, which is necessary for the formation of new bone. The addition of Ca, Mn and Zn, in particular a combination of these, to a main component in the form of MgO gives an improved effect.

Thus, in the compound according to the invention, at least 75% of the total quantity of Mg+Ca+Mn+Zn, shall be Mg, suitably in the form of MgO. Both of the metals Mn and Zn have a toxic effect at high concentrations and the concentration of these metals in the compound should not exceed 5%. Possibly, up to 10% could be acceptable. Significantly lower concentrations may be sufficient. The quantity of Ca in the compound can constitute the residual quantity up to 100%.

There are possibly other substances that can increase magnesium's stimulation of bone formation. Ca, Mn and/or Zn, however, are also suitable because adding at least small quantities of these elements to a substance intended to be placed inside the body of a person is not controversial.

In variants of the compound, Mg and Ca, Mn and/or Zn respectively, constitute sub-quantities of the total quantity of Mg+Ca, Mn and/or Zn in the compound according to the following: Mg—at least 75 percent by weight and no more than 99 percent by weight; and Ca, Mn and/or Zn—at least 1 percent by weight and no more than 25 percent by weight.

The quantity of Mn or Zn should be at least 0.01 percent by weight, preferably at least 0.1% of the total quantity of Mg+Ca and/or Zn in the compound, in order for the presence of the element or these elements to give an improved effect. The quantity of Ca should be at least 1 percent by weight of the total quantity of Mg+Ca, Mn and/or Zn in the compound.

In a suitable embodiment, the compound contains 90 percent by weight Mg, 5 percent by weight Ca, 4 percent by weight Zn and 1 percent by weight Mn of the total quantity Mg, Ca, Mn and Zn in the compound.

The compound, as mentioned above, consists of a paste or similar substance with viscous properties than can be coated on and spread over bone tissue and other surfaces of interest. The compound can thus be applied to and dispersed over bone tissue of interest by simply spreading the material on the bone tissue.

Such suitable substances can be obtained by mixing MgO powder, or another magnesium based material, with an aqueous solution such as, for example, an isotonic saline solution, or by preparing a phosphate cement mixture (which is not allowed to stiffen too much prior to application). Ca, Mn and/or Zn can be added in various ways during the manufacture. Of course, the compound must also be sterilised, of implantable quality, prior to implantation.

Preferably, the Mg is mainly present in the compound as MgO/MgOH or MgHPO₄.

The invention also concerns a compound according to the foregoing for use in the stimulation of bone formation, preferably including application of the compound on bone tissue or in marrow cavities in bone, and concerning the stimulation of compact, cortical bone. The compound can also be applied on an implant before or after the implant is placed in the body.

The invention also concerns a method for stimulating bone growth, wherein the method includes the step of applying a compound according to the foregoing on bone tissue, in bone marrow cavities or on an implant.

The invention also concerns a medical kit including a container containing the compound. The container should be of the tube type with an opening at one end of the container and a container body that is intended to be compressed in order thereby to squeeze the compound out through the opening. This “medical kit” can also include other suitable accessories such as, for example, a spatula to apply and spread the compound on intended surfaces or in intended cavities. Alternatively, the medical kit can contain two or more containers that on mixing form the specified compound. For example, this could be a two-component kit, a dry batch and a batch with liquid, in order to form a phosphate cement mixture just before application on bone tissue.

A suitable base material for the compound according to the invention is magnesium oxide, MgO, e.g. “pure MgO-powder 99,995 trace metal basis” (Sigma-Aldrich® Sweden AB, Kista, Sweden). The powder can be sterilised by heat treatment in an autoclave. The compound can be prepared in at least two ways. A paste can be prepared from MgO powder and (sterile) isotonic saline solution, and a phosphate cement can be prepared by mixing the MgO powder with (a solution of) acidic phosphate ions, such as phosphoric acid or dihydrogen phosphate. Ca, Mn and Zn can be added, for example, in the form of powders or ions in solution, before or during the preparation of the compound.

When MgO is mixed with an aqueous solution, a large proportion of the magnesium will be in the form of magnesium hydroxide, Mg(OH)ₓ.

A preparation/compound according to the invention suitably consists of 85-95 weight % Mg (in the form of MgO/MgOH or MgHPO₄), 2-13 weight % Ca, 1-5 weight % Zn and 0.1-2 weight % Mn (in the form of oxide/hydroxide or hydrogen phosphate) of the total quantity of Mg, Ca, Mn and Zn in the compound.

The magnesium based material, e.g. MgO, can be dissolved in 0.1M NaH₂PO₄ so the pH is >6.

This preparation results in a phosphate cement that, prior to stiffening, form a more or less viscous paste. The solution can include hyaluronate, approx 1-2%, to adjust the viscosity and biodegradable fibres, approx 1%, as reinforcement.

Alternately, the magnesium based material, e.g. MgO, is dissolved, e.g. in 0.15M NaCl, that is to say, an isotonic saline solution, in order to form a paste. The paste can contain approx 0.5-2% hyaluronate in order to adjust the viscosity.

The principle as such of using calcium phosphate cement for healing bone may be known, but the positive effect of a high concentration of Mg has not been noted. Such known cement is based on earth metal phosphates or hydrogen phosphates (mainly Ca, sometimes with small additions of Sr or Mg) hardening spontaneously into phosphate cement on the addition of water or saline solutions.

Thus, the compound can be used as a pharmaceutical for damaged bone tissue, in order to stimulate the formation of cortical compact bone. The compound, prepared in the form of a paste or similar substance with viscous properties, is spread on the bone tissue in question and thus provides specific local stimulation of osteogenesis in compact bone. The compound can be used in various applications, e.g. during the healing of fractures. The compound can also be used to fill in the hole after a tooth extraction.

The compound should have a dynamic viscosity in the magnitude 100s Pa, that is to say, roughly the same as toothpaste, peanut butter at room temperature or fresh, unhardened, mortar. Such a paste, that is to say, such viscous liquid, can maintain its form and position after it has been coated on and spread over the intended area. Once the compound has been applied, the paste can be allowed to harden.

The compound can also be used together with an implant, e.g. a titanium screw, in order to make the implant fasten better. The compound can be applied on the implant before it is put into place.

1-22. (canceled)

23. Compound for stimulation of bone formation, wherein the compound consists of a paste or similar substance with viscous properties that can be coated on and spread over bone tissue and other surfaces of interest, wherein the compound contains Mg and at least one of the elements Ca, Mn and Zn, and wherein Mg constitutes at least 75 percent by weight of the total quantity of Mg, Ca, Mn and Zn in the compound.

24. Compound according to claim 23, wherein Mg constitutes at least 85 percent by weight of the total quantity of Mg, Ca, Mn and Zn in the compound.

25. Compound according to claim 23, wherein Mg constitutes at no more than 99 percent by weight of the total quantity of Mg, Ca, Mn and Zn in the compound.

26. Compound according to claim 23, wherein the compound contains at least two of the elements Ca, Mn and Zn.

27. Compound according to claim 23, wherein the compound contains a mixture of Ca, Mn and Zn.

28. Compound according to claim 23, wherein Ca constitutes at least 1% and no more than 25% of the total quantity of Mg, Ca, Mn and Zn in the compound.

29. Compound according to claim 23, wherein Mn constitutes at least 0.01%, preferably at least 0.1%, and no more than 5% of the total quantity of Mg, Ca, Mn and Zn in the compound.

30. Compound according to claim 23, wherein Zn constitutes at least 0.01%, preferably at least 0.1%, and no more than 5% of the total quantity of Mg, Ca, Mn and Zn in the compound.

31. Compound according to claim 23, wherein Mg is present as MgO, MgOH or MgHPO4.

32. Compound according to claim 23, wherein the compound is sterile and of implantable quality.

33. Compound according to claim 23 for use in the stimulation of bone formation.

34. Compound according to claim 33, wherein the use includes applying the compound on bone tissue, in marrow cavities in bone or on implant.

35. Compound according to claim 33, wherein the use concerns stimulating the formation of compact cortical bone.

36. Method for stimulating bone growth, wherein the method includes the step of applying a compound according to claim 23 on bone tissue, in bone marrow cavities or on an implant.

37. Medical kit including a container containing a compound according to claim 23.

38. Medical kit according to claim 37, wherein the container should be of the tube type with an opening at one end of the container and a container body that is intended to be compressed in order thereby to squeeze the compound out through the opening.

39. Medical kit including a two or more containers containing components that on mixing together form a compound according to claim 23.

40. Method for manufacturing a compound in accordance with claim 23, wherein the method includes the step of mixing together a MgO powder and at least one of the elements Ca, Mn and Zn in an aqueous solution.

41. Method according to claim 40, wherein the method includes the step of sterilising the MgO powder, preferably by heat treatment in an autoclave.

42. Method according to claim 40, wherein the method includes the step of adding hyaluronate to the mixture in order to adjust the viscosity.

43. Method according to any of the claim 40, wherein the aqueous solution is an isotonic saline solution.

44. Method according to any of the claim 40, wherein the aqueous solution contains acid phosphate ions.
